(19) United States
(12) Patent Application Publication
(10) Pub. No.: US 2024/0260877 A1
(43) Pub. Date: Aug. 8, 2024

(54) IMPEDANCE TOMOGRAPHY

(71) Applicant: CYQIQ LTD, London (GB)

(72) Inventors: David Holder, London (GB); Kirill Aristovich, London (GB)

(21) Appl. No.: 18/567,083

(22) PCT Filed: Jun. 8, 2022
(86) PCT No.: PCT/GB2022/051432

§ 371 Date: Dec. 5, 2023

(30) Foreign Application Priority Data
Jun. 11, 2021 (GB) 2108374.6

(51) Int. Cl. A61B 5/246; A61B 5/00; A61B 5/0536

(52) U.S. Cl. CPC A61B 5/246; A61B 5/0536; A61B 5/4064; A61B 5/6803; A61B 2562/0223

(57) ABSTRACT

A non-invasive method of determining electrical properties within the brain of a human or animal subject is disclosed. Electrodes are disposed across the scalp of the subject, and atomic magnetometer sensors are disposed around the scalp. Then, for each of a plurality of combinations of the electrodes, a probe electrical signal is applied to the electrodes of the combination and magnetic field signals arising from the probe electrical signals are measured at each of a plurality of the atomic magnetometer sensors. The measured magnetic field signals may then be used to determine electrical properties within the brain.

The present invention relates to methods and apparatus for determination of electrical properties in a human or animal subject, or for determination or measurement of other properties or aspects of the subject using such electrical properties.

Using the invention, electrical properties or activity with the brain, or indeed in other parts of the subject may be measured non-invasively, and such measurements may be used to determine, measure, or image aspects of the subject such as fast neuron activity, blood flow changes, or pathologies such as stroke or tumour.

### INTRODUCTION

Non-invasive biomedical electrical impedance tomography (EIT) is typically undertaken using ECG-type electrodes placed around a body part of interest such as the torso including the lungs and heart. Probe electrical signals, typically as alternating currents of known magnitude, are applied sequentially to different pairs or groups of electrodes, and the resulting voltages at some or all of the electrodes are recorded. These measurements are then used to reconstruct an image or map of impedance or equivalent electrical properties of the body between the electrodes, for example as a cross-section through the torso.

Various attempts have been made in the prior art to measure electrical properties within the brain using electrical impedance tomography, but for non-invasive techniques the high impedance of the skull significantly lowers both the available signal to noise ratio and the resolution of the reconstructed image.

It would be desirable to address problems of the related prior art.

### SUMMARY OF THE INVENTION

The invention provides methods and apparatus for carrying out impedance tomography on human or animal subject, comprising applying probe electrical signals to the subject using a plurality of electrodes, such that imposed electrical currents flow within the subject, and detecting magnetic fields arising from the imposed electrical currents using a plurality of magnetometers disposed about the subject, and typically dispersed or arranged to cover over the same or a similar area of the subject as the electrodes. The described techniques may be referred to as magnetic detection electrical impedance tomography, or MD-EIT.

In particular, atomic magnetometers may be used, for example optically pumped magnetometers, since these do not require any cryogenic cooling, and are available in sufficient compact form to locate in suitable numbers around the scalp or other body part, in combination with a similar number of electrodes.

More specifically, the invention provides methods and apparatus for imaging brain function non-invasively, by using impedance tomography (or electrical impedance tomography, EIT, or more particularly magnetic detection electrical impedance tomography MD-EIT) and atomic magnetometers.

Impedance tomography images or maps of electrical properties such as conductance or impedance, in two or three dimensional form, may then be formed using established principles for EIT reconstruction, for example using inversion of a Jacobian sensitivity matrix based on linear assumptions.

The resulting maps or images may for example be of fast neural impedance changes relating to ion channel opening, of slower blood flow related changes over several seconds or so, or of stroke or other intracranial pathologies.

For fast neural changes, using probe electrical signals at around 1.7 kHz, or around 2 kHz, for example between around 1 and 2.5 kHz, it may be practical to avoid the need for a magnetically shielded chamber and so provide apparatus uniquely able to produce images of circuit activity in the brain in a non-specialist environment for example by paramedics or ambulance crews, with the magnetic sensors and/or electrodes mounted in a convenient helmet or cap structure. For imaging slow blood volume changes related to cerebral activity or pathology such as a stroke, this would offer a portable device able to produce images within seconds to minutes, even in remote locations, which could be used to inform medical practice.

In particular, the invention provides a non-invasive method of determining electrical properties within a human or animal subject, for example within the head or brain, comprising: disposing a plurality of electrodes across the scalp (or other body region) of the subject; spacing a plurality of atomic magnetometer sensors around the scalp (or other body region) of the subject; for each of a plurality of combinations of said electrodes, applying a probe electrical signal to the electrodes of said combination and measuring magnetic field signals arising from the probe electrical signals at each of a plurality of the sensors; and using the measured magnetic field signals to determine electrical properties within a region between the electrodes, whether that is the brain or some other body region.

So although the head or brain may be probed or imaged using the described techniques and apparatus, these may instead be used to probe or image other body parts such as the neck, chest, thorax, hips, limbs, lungs, and so forth, as well as regions, structures, and organs within these and other body areas such as the spine, peripheral nerves, the lungs and so forth.

The atomic magnetometer sensors may be optically pumped magnetometer sensors, or other atomic magnetometer sensors not requiring cryogenic cooling. At least sixteen atomic magnetometers may be used. The surface of each atomic magnetometer sensor facing the scalp or other body surface, or the active sensing region of the sensor, may be positioned so as to be spaced no more than 2 cm, or no more than 1 cm, from the surface of the scalp or other body part.

To this end, the atomic magnetometer sensors may be mounted to a helmet or cap configured to fit on or around the scalp, and optionally the electrodes may also be mounted to the cap.

Each probe electrical signal may have a frequency, or principal frequency, or tone, in the range from 100 Hz to 15 kHz, or in the range from 1.0 to 2.5 kHz, or in the range 1.4 KHz to 2.0 KHz. Such frequency ranges are found to be useful for determining electrical properties which comprise properties representing neuronal depolarisation or other fast neural changes within the brain, and to this end the methods may comprise measuring or detecting neuronal depolarisation within the brain using probe electrical signals within such frequency range, and optionally making such measurements with a time resolution of less than 100 milliseconds, or less than 20 milliseconds.

In order to measure fast neural changes, the method may further comprise providing a repeated stimulation to the human or animal subject, and for each stimulation applying a probe electrical signal to one or more of the combinations of electrodes, and measuring the magnetic field signals arising from the applied probe electrical signal. Magnetic field signals from the full range of electrode combinations being used can then be built up over multiple repeat stimulations.

More generally, Each probe electrical signal may have a frequency in the range from 100 Hz to 100 kHz. Probe electrical signals in this frequency range may be used to determine said electrical properties with a time resolution of from 0.1 to 10 seconds, or from 0.5 to 5 seconds. In this regime, aspects of the determined electrical properties might for example be evoked electrically, for example to present spontaneous normal functions or epileptic seizure. Probe electrical signals may then be applied to two or more of said combinations of electrodes simultaneously, the simultaneous probe electrical signals having different frequencies, and the method then comprises distinguishing the resulting magnetic field signals with reference to the different frequencies of probe signals, in a frequency division multiplexing technique.

Operating with probe electrical signals of frequencies above about 1 kHz can also have particular advantages in reducing the effects of magnetic noise, for example ranging from the Earth's static field through to nearby electrical equipment, cables, machinery and moving objects.

The methods may further comprise carrying out a tomographic inversion of the magnetic field signals to determine a map or image of the electrical properties. The tomographic inversion may be carried out with reference to or using the probe electrical signals, typically with knowledge of the generated signals, although actual measurements of the probe electrical signals could be used.

Typically, the electrical properties determined are impedance or measures of impedance, or equivalently conductance or similar measures, although other derived measures may be determined such as admittivity, dielectric constant, or changes or rates of change in any of the aforementioned measures.

The invention also provides method of imaging or detecting fast neuron activity, blood flow changes, epileptic seizure, pathologies such as stroke or tumour, or other aspects described in this document, within the brain of a human or animal subject, by using the detected or measured magnetic fields, and/or by using the determined electrical properties.

The invention also provides apparatus for carrying out the methods mentioned above and elsewhere in this document, for example apparatus for carrying out non-invasive determination of electrical properties within a human or animal subject, for example within the brain or head or other body part, comprising: a plurality of electrodes for applying probe electrical signals to the scalp (or other body part) of the subject; a plurality of atomic magnetometer sensors arranged to fit around the scalp (or other body part) of the subject so as to measure magnetic field signals arising from the probe electrical signals; and a probe signal source arranged, for each of a plurality of combinations of said electrodes, to apply a probe electrical signal to the electrodes of said combination.

The apparatus may further comprise a reconstructor or tomographic inverter arranged to carry out a tomographic inversion of the measured magnetic field signals to determine the electrical properties.

In particular, the atomic magnetometer sensors may be optically pumped magnetometer sensors. The atomic magnetometer sensors may be arranged so as to be spaced no more than 1 cm from the surface of the scalp, or in other ways as discussed above.

The apparatus may further comprise a helmet or cap configured to fit on the scalp, wherein each atomic magnetometer sensor is mounted to the cap, and optionally wherein the electrodes are also mounted to the cap.

### BRIEF DESCRIPTION OF THE DRAWINGS

Embodiments of the invention will now be described, by way of example only, with reference to the drawings, of which:

FIG. 1 illustrates schematically apparatus for carrying out non-invasive electrical impedance tomography on a human or animal subject, using magnetic field sensors;

FIG. 2 illustrates in more detail how various aspects of the electronics and software of FIG. 1 may be implemented; and

FIG. 3 provides a flow chart of a method according to the invention.

### DETAILED DESCRIPTION OF EMBODIMENTS

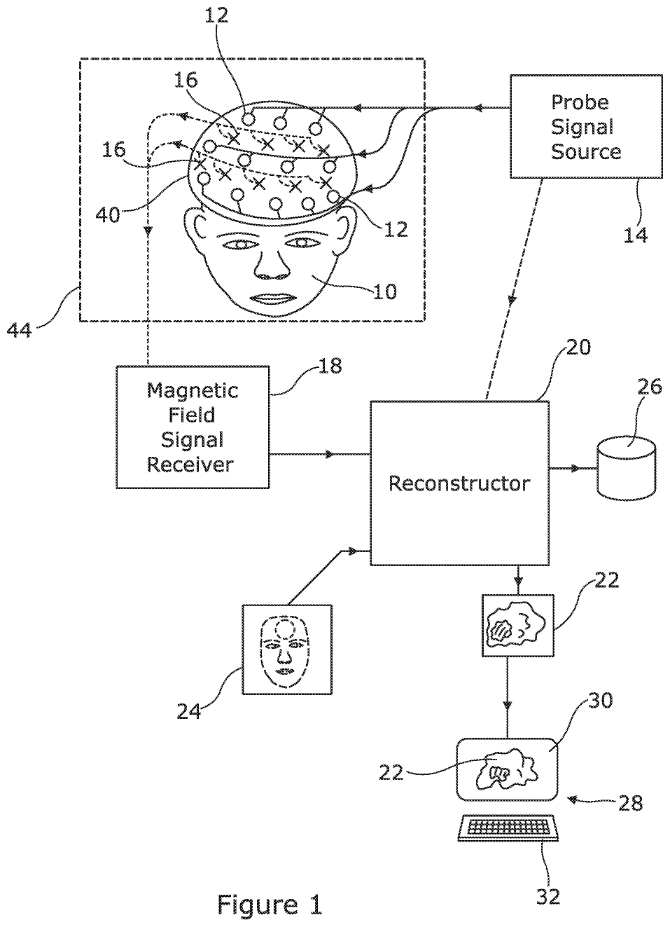

FIG. 1 shows schematically apparatus for carrying out non-invasive determination of electrical properties within tissue of a human or animal subject 10, for example within the head or brain of the subject. To this end, an array of electrodes 12 is disposed across and coupled to the skin of the subject, for example across and electrically coupled to the scalp of the subject as shown in FIG. 1, in which the electrodes are shown using the “O” symbols. Because of the magnetic mode of detection used, the described techniques may be referred to as magnetic detection electrical impedance tomography.

Each of the electrodes 12 is coupled to a probe signal source 14. The probe signal source 14 is arranged to provide probe electrical signals to the electrodes 12 for coupling into the tissue of the subject. In particular, to provide a variety of different current paths through the tissue of the subject 10, the probe signal source 14 is arranged, for each of a plurality of different combinations of the electrodes, to apply a probe electrical signal to the electrodes of that combination. Different ways in which these signals may be applied, for example in sequence for each combination, or using multiplexing techniques, and the nature of these signals for example in terms of alternating current frequency, are discussed in more detail below.

The applied probe electrical signals give rise to imposed electrical currents flowing within the tissue of the subject 10, with the detailed distribution of these currents depending on the distribution of conductivity or impedance within the tissue. The imposed electrical currents in turn give rise to corresponding magnetic field components which can be detected as magnetic field signals.

An array of magnetic field sensors 16 (shown in the figure as “X”) is also disposed across the subject, i.e. across the scalp in FIG. 1. The electrodes 12 and magnetic field sensors 14 may typically be interspersed, although note that the magnetic field sensors do not need to be in contact with the scalp or other skin of the subject, and in principle sensors and electrodes can overlap or be co-positioned with respect the scalp, or can be located in different regions of the scalp. Generally, however, the area of the body over which the electrodes are dispersed will be largely overlapping, for example at least 50% or at least 80% overlapping, with the area of the body over which the magnetic field sensors are dispersed.

The magnetic field sensors are arranged to detect the magnetic field signals arising from the imposed electrical currents, and to pass these signals to a magnetic field signal receiver 18.

The magnetic field signal receiver 18 passes the magnetic field signals, which as noted above contain information about the imposed electrical currents, to a reconstructor 20 which uses the magnetic field signals, and their relationship to the probe electrical signals giving rise to the imposed electrical currents (for example using information received from the probe signal source 14, or from measurements of the probe signals), to construct a map or image 22 of electrical properties of the tissue under examination, typically using tomographic inversion. When we refer to such a map or image this is not generally intended to require the graphical display of such an image to a user, although of course such display may take place, but rather to the array of data which represents the electrical properties determined across a slice or volume of the subject tissue.

Since the distribution of imposed electrical currents within the tissue is related to the distribution of conductivity (or impedance), the electrical properties determined by the reconstructor 20 may typically be conductivity (or impedance) of the tissue through which the imposed currents pass, or a similar measure. Although the reconstructed image 22 may represent a two dimensional section through the brain, comprising a two dimensional array of pixels, more typically the image 22 will be three dimensional in nature, formed of a three dimensional array of voxels each representing a volume.

In some embodiments, however, it may be sufficient to recover more limited electrical property data, for example at a single point or averaged over a particular region. In such cases it may not be necessary to use a full, or indeed any, tomographic inversion to recover the desired electrical properties of the subject tissue, with some other more direct or limited data analysis being used instead. Note however, that due to the complex relationship between the probe electrical signals, the imposed currents and the resulting magnetic field signals, at least some limited tomographic inversion will usually be required.

Embodiments of the invention may be used to determine absolute values, maps or images of electrical properties such as conductance or impedance, but frequently it will be small changes over time in such electrical properties which will be of principal interest, for example localised changes in conductivity caused by neuronal depolarisation which typically takes place on time scales of tens of milliseconds, or localised changes in conductivity caused by changes in blood flow which typically take place on time scales of a few seconds. Reconstructed maps or images of electrical properties within the tissue may therefore refer either to absolute values of such properties, changes over time in such properties, or indeed changes in time and space, gradients, and other derived measures, or combinations of these.

The reconstructor 20 typically uses a base model 24 of the tissue, for example of a subject head and brain, in order to support the image reconstruction. This base model 24 may be generic to multiple subjects and of level of detail varying from simplistic to anatomically detailed, but advantageously the base model 24 may be derived from measurements of the actual human or animal individual under examination, for example being derived from MRI imaging, CT imaging, or other imaging techniques applied to the subject, and/or from measured dimensions.

The magnetic field signals, image 22, and/or other related data may be stored for future use for example at storage 24, and may be displayed or used for display for example at a local or distant computer terminal 26. Computer terminal 26 is depicted as being provided with a display 28 and suitable control means 30 such as a keyboard and/or a mouse, such that the image 22 may be displayed and manipulated by a user. For example if the image is a three dimensional image of the brain, it may be rotated, sectioned, contoured and so forth to permit better study and clinical or research use.

The electrodes 12 used to apply the probe electrical signals to the tissue of the subject may conveniently be conventional EEG (electroencephalogram) or similar electrodes which typically include an adhesive pad for holding the electrode onto the skin of the subject, for example the scalp, and an exposed metallic electrode at the centre of the adhesive pad. However, for convenience and in order to ensure accurate placement and positioning of electrodes, both in absolute terms and relative to each other, the electrodes, and also the magnetic sensors, may be mounted to a cap 40, or similar structure. In FIG. 1 the cap 40 is shown as worn on the head of the subject, but other structures may be used for other parts of the body.

The cap may preferably be rigid in order to ensure more accurate electrode placement, and if required may be formed specifically to fit the head of a particular individual subject. If mounted to the cap 40, the electrodes need not be adhesive. Instead, for example, each electrode could be telescopic or sprung in other ways so as to press down onto the scalp of the subject.

The total number and placement positions of the electrodes may vary depending on need, for example, to optimise for particular resolutions of final image 22 and for particular regions of the subject tissue, but typically between about 20 and 80 electrodes may be used, for example 32 electrodes, with the electrodes being approximately evenly spaced across the scalp or cranium, or other body region being examined. A typical spacing between electrode centres may therefore be around 3 to 5 cm.

In order to provide a sufficient diversity of current paths to permit image reconstruction, the probe signal source is arranged to apply probe electrical signals to each of a plurality of different combinations of the electrodes, and the resulting magnetic field signals are measured for each such electrode combination. Typically, each combination will consist of just two electrodes. The total number of different combinations may be limited in order to constrain the time and/or multiplexing channels required to complete the full set of combinations. For example, if thirty two electrodes are used then the number of different combinations of electrodes used for applying separate probe electrical signals may also be 32 or thereabouts. The distinct combinations may be chose in various ways to optimize the reconstructed image of the electrical properties of the tissue, for example as discussed in Andy Adler et al. 2011 Physiol. Meas. 32 731, https://pubmed.ncbi.nlm.nih.gov/21646709/.

The probe electrical signals will typically give rise to sufficiently small imposed current levels for safe clinical use, for example around 1 milliamp, which can easily be achieved for example as described in O. Gilad et al., “Design of electrodes and current limits for low frequency electrical impedance tomography of the brain”, Med. Biol. Eng. Comput., 45, 7, 621-633, 2007. Probe electrical signals at or close to DC can be used in principle, for example a square wave signal with a cycle length of around a second as described in O. Gilad et al., Neuroimage 47 (2009) 514-522. However, more typically the probe electrical signals will be of a frequency, or principal frequency, which can vary according to the processes or characteristics of the subject tissue which are to be examined. These signals may be described as AC, although it may not be necessary for the signal to alternate across the zero current or voltage level.

The probe signal source may provide probe electrical signals which are effectively voltage signals (voltage constrained to oscillate at the desired frequency with a chosen voltage range or RMS value) or current signals (current constrained to oscillate a the desired frequency with a chosen current range or RMS value). In these two situations the probe signal source could be described as a voltage source or current source respectively. In either case, suitable currents are imposed within the tissue and lead to magnetic field signals which can be used by the reconstructor 20. Reasons for and consequences of choosing either a voltage source or current source arrangement are discussed in more detail below.

Typically the frequency of the probe electrical signals may be of the order of around 100 Hz to around 100 kHz. To measure fast changes in the tissue on time scales of milliseconds to tens of milliseconds, considerable bandwidth in the detected magnetic field signals is required, and it may be preferable to apply a probe electrical signal to just one combination of electrodes at a time. However, for the detection of slower processes on time scales of seconds, or of essentially static electrical properties, which do not require large bandwidth in the detected magnetic field signals, it may be desirable to apply probe electrical signals to multiple combinations of electrodes at the same time, or even to all combinations at the same time, using a multiplexing technique. Typically, a frequency division multiplexing technique may be used wherein probe electrical signals are applied to two or more of the combinations of electrodes simultaneously, the simultaneous probe electrical signals having different frequencies, and the resulting magnetic field signals are distinguished using related frequency bands. The width of the required frequency bands for the magnetic field signals around the principal frequency of the probe signal will depend for example on the time scales of changes which are to be measured in the electrical properties of the tissue.

If each probe electrical signal leads to an imposed current of the order of 1 milliamp in magnitude flowing through the tissue between a pair of the electrodes 12, the resulting magnetic field components external but close to the human or animal subject will typically be of the order of 100 picoTesla, and around this level in particular when the electrodes are in contact with a human scalp and the tissue is the brain, although the magnetic field levels will of course differ somewhat for much larger or smaller body regions under examination. Impedance changes within brain tissue are of the order of 0.1% for neuronal depolarisation, or of the order of 1.0% for blood flow changes, and if these take place within a volume of the brain with a diameter of around 1-2 centimetres, the resulting changes to the above magnetic field components close to the scalp are of the order of about ten to a hundred femtoTesla, with these changes being relatively larger for impedance changes close to the surface of the brain, and relatively smaller for impedance changes deep within the brain.

It is possible to measure such magnetic fields and magnetic field changes using SQUID sensors (superconducting quantum interference devices), but these require cooling to very low temperatures, therefore also requiring cryogenic Dewar containment and a resulting spacing of the SQUID detector element from the surface of the scalp of several centimetres. This in turn leads to a significant reduction in magnitude of the measured magnetic field signals. In embodiments of the invention therefore, and as illustrated in FIG. 1, each magnetic sensor 16 may be provided using an atomic magnetometer, for example an optically pumped magnetometer.

Atomic magnetometers typically operate substantially at room temperature rather than requiring a cryogenic environment, although the small gas cell typically found at the heart of the device is usually heated to 100° C. or more. Moreover, they are commercially available in compact packages with an end face of the housing behind which the detection point lies having a footprint of around 2 to 5 cm², allowing the magnetic field sensors to be interspersed easily between the electrodes across the scalp or other part of the subject to be examined.

Atomic magnetometers are also commercially available which have a small standoff distance from the end face of the housing to the magnetic field sensing point within the sensor, for example of only a few millimetres, permitting detection of magnetic field signals very close to the scalp.

Such atomic magnetometers typically operate through interaction of the atomic spin of atoms of a vapour held within a gas cell, with both a pump laser beam and the magnetic field to be sensed, with detection of the magnetic field typically being realised by some effect such as transmittance, absorption, or polarisation of either the pump laser beam or a separate probe beam. Alkali metal atoms are typically used in the gas cell for the spin interactions, although other buffer species may also be added. Such atomic magnetometers may in particular use the pump laser to spin polarize the atoms via a process of optical pumping, and may then also be described as optically pumped magnetometers, which include devices such as SERF (spin exchange relaxation-free) magnetometers in which the rate of spin-exchange collisions greatly exceeds the Larmor precession frequency. Suitable optically pumped magnetometers for use as magnetic sensors in embodiments of the present invention are provided by QuSpin, Inc. of Louisville, USA, see http://quspin.com, and include the QZFM Gen-2 zero-field magnetometer.

The number of magnetic sensors 16 provided may typically be of the same or similar to the number of electrodes 12, for example with 32 electrodes and 32 magnetic sensors. Using a much larger number of magnetic sensors than electrodes is unlikely to increase the useful resolution of the image 22 significantly, while greatly increasing cost and the computational expense of forming the image 22. Using significantly fewer magnetic sensors 16 than electrodes 14 would likely impact on the useful resolution of the image 22.

The magnetic sensors 16 and the electrodes 14 may be largely interspersed across and around the part of the human or animal subject to be examined, for example across the scalp in the example of FIG. 1, and will typically be mounted to a cap 40 or similar structure also as shown in FIG. 1, and as already described above in respect of the electrodes. As noted above, if around thirty two electrodes are used, the spacing between electrode midpoints will be around 3 to 5 cm, and so if a similar number of magnetic sensors 16 are used, and are distributed across the same area of the human or animal subject as the electrodes, these will also have a midpoint spacing of around 3 to 5 cm.

Each magnetic sensor 16 is arranged so as to be able to detect the magnetic field signals arising from the imposed electrical currents within the tissue. If changes in the determined electrical properties over time are to be detected, then each magnetic sensor also needs to be sufficiently sensitive such that the related changes to the magnetic field signals can be detected.

Typically, each magnetic sensor 16 may be used to measure the local magnetic field signal at the sensor in three dimensions, i.e. as a three dimensional vector. However, in some embodiments it may be sufficient to measure a two dimensional magnetic field vector within a plane, a magnetic field strength in one or more particular directions, of a magnetic field scalar value, at least for some of the magnetic sensors for example depending on position in the array of such sensors. Commercially available atomic magnetometers are generally able to measure magnetic field properties in three dimensions if required, including the QuSpin sensor mentioned above.

Atomic magnetometers are typically configured to measure absolute magnetic field strengths, and so the magnetic field signals will typically represent such absolute field strengths. However, embodiments of the invention may be implemented where the magnetic field signals represent magnetic field gradient at each sensor, or other parameters such as higher order moments or time derivatives of field, gradient or moments.

In order to minimise noise due to stray or background magnetic fields, at least the body part of the subject being examined, along with the electrodes 12 and magnetic field sensors 16, may be located within a magnetically shielded chamber 44, which optionally may also contain the probe signal source 14, magnetic field signal receiver 18, and optionally other parts of the apparatus. Indeed, the apparatus and subject may be placed in a magnetically shielded room if required. However, additionally or alternatively, various measures to reduce magnetic noise and the effect of background variations in the magnetic field may also be implemented within the magnetic field sensors themselves. For example, many atomic magnetometers may already comprise electrical coils for applying magnetic fields to the sensor for the purposes of implementing the magnetic field measurements, and these electrical coils may also be used for nulling background magnetic fields and controlling other noise.

However, although active cancellation of unwanted magnetic fields using coils in the magnetometers of elsewhere, or passive cancellation using shielded chambers or rooms may be used, either or both of these may be avoided by use of probe electrical signals within certain frequency ranges, for example above about 1 kHz, so that the effects of both the Earth's static magnetic field, and of main components of magnetic noise for example from power cables, power supplies and nearby electrical equipment and moving metal objects, are avoided.

For example, as discussed elsewhere in this document, using probe electrical signals round 1.7 kHz, or between around 1.0 kHz to 2.5 kHz, including for detection of fast neural changes may both provide advantages in terms of sensitivity to those neural changes, and in terms of reduction of magnetic field noise.

As touched on above, the probe signal source may be arranged to deliver probe signals essentially as a current source, so that the probe signals have substantially constant current range or RMS current, or as a voltage source, so that the probe signals have substantially constant voltage range or RMS voltage.

In principle, implementing the system using a constant voltage approach might be expected to provide a greater change in magnetic field signals for a given change in impedance, since the imposed currents would be more free to respond to impedance changes. However, the inventors carried out a detailed simulation of the described methods on an anatomically realistic 35 million finite element model of a human head with 32 electrodes and 500 magnetic field sensors. Simulated probe electrical signals of approximately 1 mA at 1.5 kHz were applied and a disturbance to be detected was modelled as neural depolarisation activity having a spherical 1% local impedance decrease, with a 2 cm diameter, compared to the surrounding brain tissue.

As expected, the constant voltage probe electrical signals provided the better signal to noise ratio in detection of the disturbance, but the improvement over use of a constant current probe electrical signals was only of the order of 6%. This difference is unlikely to be sufficient to make implementation of the described embodiments using constant voltage sources worthwhile if this requires extensive redesign of the constant voltage probe signal sources more commonly already used for conventional biomedical electrical impedance tomography.

However, using the same model the inventors demonstrated that the signal to noise ratio of the detected disturbance was roughly 150% greater than that obtained using the same 32 electrodes for a more conventional electrical impedance tomography approach in which the disturbance was detected by measuring voltages at the electrodes instead of by measuring the magnetic field signals.

This increase in sensitivity was based on a sensitivity of magnetic field detection of 245 femtoTesla at 600 Hz bandwidth. It is expected that the sensitivity of available atomic magnetometers will increase considerably further over coming years making the described techniques using magnetic field sensing for biomedical electrical impedance tomography even more attractive in terms of sensitivity.

The reconstructor may be arranged to use any of a variety of known tomographic inversion or reconstruction techniques, for example using inversion of a Jacobian sensitivity matrix based on linear assumption with regularisation. Various other suitable techniques are described for example in Vauhkonen M. et al., IEEE transactions on Biomedical Engineering, vol. 45, page 486. This document describes algorithms for electrical impedance tomography reconstruction based on the formulation of EIT as a state-estimation problem, using a Kalman filter for recursive estimation of state. Although electrical impedance tomography inversions usually rely on measured voltage patterns resulting from imposed probe signals, essentially the same inversion or reconstruction techniques can be used in the present case where measured magnetic field signals are instead used. Essentially, the Jacobian matrix can be computed using the same algorithms and software as typically used for conventional electrical impedance tomography, with the additional step of computing the magnetic fields using the Biot-Savart law. Tomographic inversion algorithms and software used can then be the same as those used for conventional electrical impedance tomography, for example utilizing 0-th order Tichonov regularisation, or an iterative Gauss-Newton method.

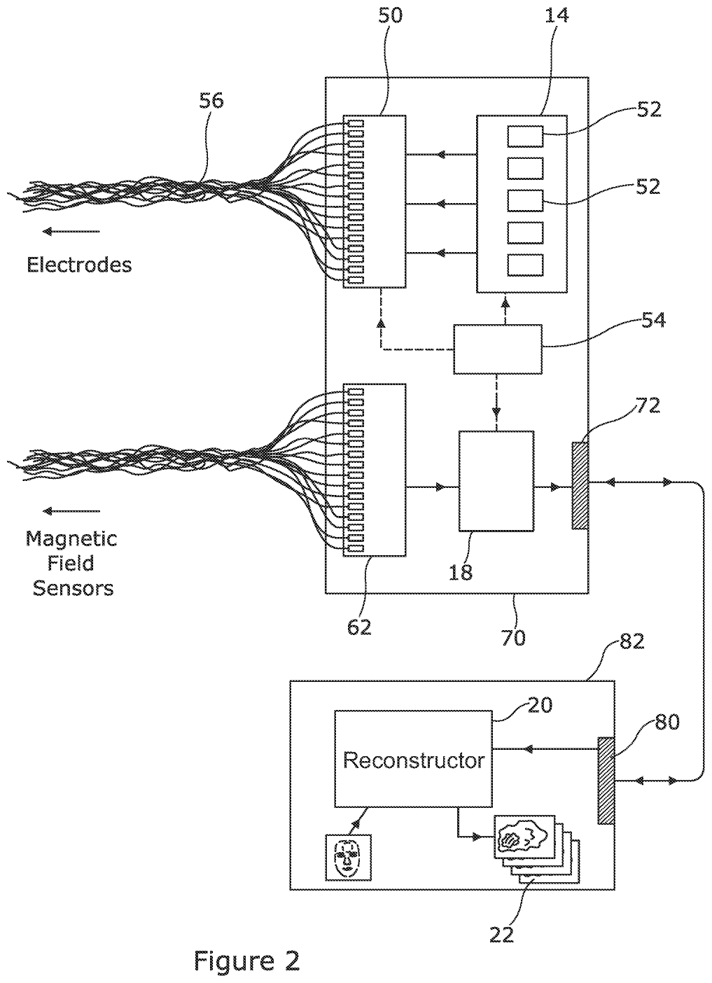

The apparatus, control, probe signal generation, magnetic field signal collection and data processing functionality depicted in FIG. 1 and described above can be implemented and distributed in various ways. One such way is depicted in FIG. 2 in which the probe signal source 14 is coupled to the electrodes via a probe switch 50. The probe signal source 14 may typically comprise one or a plurality of separate voltage or current sources, each arranged to provide probe electrical signals as discussed above. For example a separate voltage source 52 could be arranged to generate a probe electrical signal having one of several different signal frequencies being used. The probe switch 50 is then arranged to couple the correct probe electrical signals to the electrodes with appropriate timings under the control of controller 54, through electrode leads 56. The controller 54 may also control aspects of the probe signal source such as the principal frequencies of each of the one or more voltage or current sources.

Magnetic field signals received from the magnetic field sensors in electrical form (typically digitally) through sensor leads 60 may be received at a sensor switch 62 for combining or multiplexing the signals into a single stream, and the magnetic field signals are then passed to the magnetic field receiver 18 for storage, buffering or similar in preparation for use in image or map reconstruction. Since the magnetic field sensors will typically all transit magnetic field signals continuously, and not all of the signals may be required all of the time, the magnetic field receiver 18 may selectively store particular parts of these signals, for example under direction from the controller 54.

The above components may conveniently be contained within a single driver housing 70, which is connected to the electrodes and sensors by the various leads, with the electrodes and sensors optionally mounted to a rigid cap 40 for wearing on the subjects head, or other rigid or more flexible carrier for the electrodes and sensors. The describes components and functions may be implemented in a mixture of electronic component hardware, and software, for example with the controller implemented using a microcontroller and the switches using programmable switch components.

The reconstructor 20 may also be housed within the driver housing 70, but since the tomographic inversion process is computationally expensive, typically requiring significant processing power, the reconstructor 20 may more conveniently by implemented in software using one or more computer processors external to the driver housing, optionally located distantly from the driver housing, for example in a laptop, remote server unit, etc. To this end, FIG. 2 shows a data stream 74 transmitted from a network connection 72 in the driver housing 70 to a network connection 80 of an analysis server 82 such as a laptop, within which the reconstructor 20 is implemented in software. Also provided within memory of the analysis server 82 is the base model 24 of the tissue of the subject, used for constraining the tomographic inversion as already mentioned above, and an output map or image of electrical properties of the tissue being imaged, typically a three dimensional map, or for example a series of such maps forming multiple frames within a time series. A display of the analysis server 82, or some other display, can then be used for display of the generated maps or images of the human or animal subject for example as already depicted in FIG. 1.

The analysis server 82 may comprise one or more microprocessors, suitable program and data memory and input/output mechanisms for implementing the reconstructor, as well as other functionality, in software. Such software may also provide a user with control of the elements in the driver housing, for example via the network connection shown in FIG. 2.

As touched on already above, the described methods and apparatus may be used to measure fast brain activity such as neuronal depolarisation within the brain of a human or animal subject. Such fast brain activity typically takes place over a few to a few tens of milliseconds, as ion channels in the neurons open and lead to an increase in local conductivity, and the invention also comprises methods measuring such activity using the determined electrical properties.

The inventors have found that using probe electrical signals with a frequency of around 1.7 kHz, or more generally from around 1.0 KHz to 2.5 kHz, or more generally still from around 100 Hz to 15 kHz, provides various benefits as discussed in more detail below. Typically the duration of the probe electrical signal for each separate electrode combination may be around 0.1 to 1.0 seconds. Because the conductivity changes which are sought to be measured occur quickly, considerable bandwidth in the detected magnetic field signals is required, of the order of 200 Hz to 1 kHz, so that using multiple probe electrical signals on multiple electrode combinations at the same time with frequency division multiplexing may not be practice.

Instead, it may be desirable to apply a probe electrical signal to each electrode combination in turn to achieve the desired bandwidth. This can be implemented by providing a regularly repeated stimulation to the human or animal subject, and for each repeated stimulation applying a probe electrical signal to one of the combinations of electrodes and measuring the magnetic field signals arising from the applied probe electrical signal. Over time, magnetic field signals relating to all combinations of electrodes are collected, each relating to near identical stimulation, so that these can be combined into a single image or map of the electrical response to the stimulation, or more useful a single series of images of maps having a number of frames spanning the duration of a single probe signal, for example about 1.0 seconds.

If thirty two different electrode combinations are used in sequence, each for a 0.5 second window, and the series of combinations is then repeated 32 times to improve signal to noise ratio of the resulting magnetic field signals and reconstructed image or map, then it will take about eight minutes to collect the data required to reconstruct a single image or map for a short time period within the 0.5 second window, which could represent a time point within the window of about 10 milliseconds, or if required a series of such images or maps such as a series of 500 image frames within the window.

Maps or images may represent actual or absolute electrical properties such as conductivity at a particular time point (for example a 10 millisecond window), or a change properties from one time point to the next, or some other measure of change.

The choice of repeated stimulation will depend on what particular measurement or study of the subject is required, but typical stimulations may be a brief display of a checkboard pattern in the subject's field of view, or momentary press of an activator on a fingertip.

In order to detect or image slower changes in brain conductivity, for example due to changes in blood flow which typically take place over periods of a few seconds, or changes during epileptic seizures, a smaller bandwidth of detected magnetic field signals is required, so that multiple probe electrical signals of different frequencies can be applied to the subject tissue at the same time in a frequency divisional multiplexing scheme. In some such arrangements all of the different combinations of electrodes may be used simultaneously, for example with thirty two different frequencies of probe signals applied simultaneously to thirty two different electrode combinations. In this way, magnetic field signals for generating a full image or map frame can easily be collected in between, say, 0.1 and 1.0 seconds, so that a series of images of maps in close to real time can be generated.

A typical set of probe signal frequencies could be at spacings of 100 Hz from about 1 kHz to about 4 kHz. However, more generally probe electrical signals for detecting slower changes in the brain or other tissue could have principal frequencies within from about 100 Hz to about 100 kHz, and could use no frequency multiplexing, full frequency multiplexing with all probe electrical signals being used together, or a lesser degree of frequency multiplexing, for example with four or eight different probe electrical signals being used together.

Probe electrical signals in these frequency ranges may be used to determine said electrical properties with various time resolutions for example from about 0.1 to 10 seconds, or from about 0.5 to 5 seconds. In this regime, aspects of the determined electrical properties might for example be evoked electrically, for example to represent spontaneous normal brain functions or epileptic seizure. The determined electrical properties map also or instead comprise properties representing blood flow changes for example related to cerebral activity or pathology such as stroke. The invention therefore also provides a method of measuring any of these aspects using the determined electrical properties

The described methods and apparatus can also be used to image essentially static conductivity features, for example in examining stroke conditions, brain tumours and other pathological conditions. Since the features being imaged in this case do not significantly change over periods of at least minutes, longer integration times may be used if desired to further improve image quality. To this end, the invention also provides a method of measuring or detecting such pathologies such as stroke or tumour using the determined electrical properties.

Various non-linear reconstruction methods may be used in determining the maps or images of tissue in these circumstances. For example Bayesian techniques, or machine learning such as deep learning techniques may be used. Maps or images of absolute impedance may be constructed using probe electrical signals of either a single frequency or of multiple different frequencies, or maps or images of impedance spectra with respect to frequency may be constructed using probe electrical signals at multiple different frequencies.

Although FIGS. 1 and 2 show various apparatus features of embodiments of the invention, those figures have also been used to describe methods of use and procedures to obtain maps or images of electrical properties of the studied tissue.

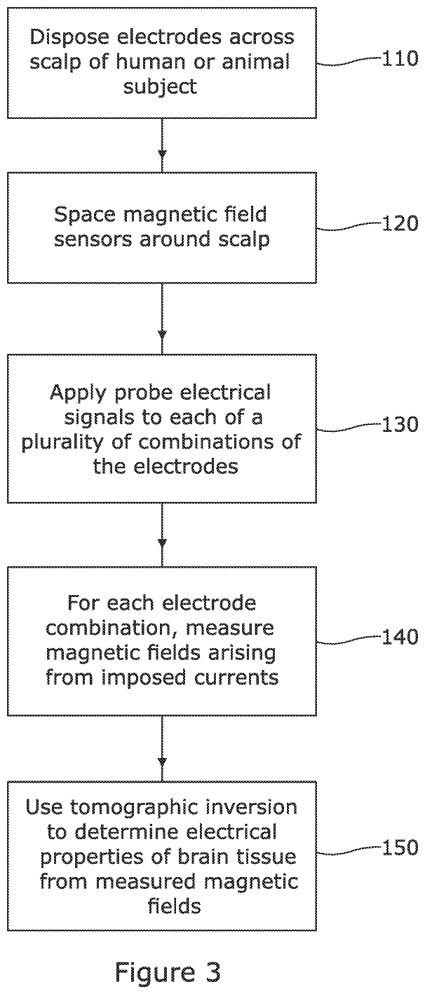

FIG. 3 more particularly illustrates methods embodying the invention, which can be implemented using the described apparatus, to provide non-invasive measurement of electrical properties of tissue of a human or animal subject, for example within the head or brain.

In step 110, electrodes are disposed across the scalp of a human or animal subject (or across another body area as desired). Typically around 32 electrodes might be used, spaced fairly evening for example with typical centre point spacings of around 3 cm-5 cm. The electrodes may mounted to a cap or other structure to assist accurate positioning and to avoid unwanted movement while in use.

In step 120, magnetic field sensors are also placed around the scalp. These need not be in contact with the scalp, but should be placed as close as practical to maximise the magnetic signal detected from within the head of the subject. Atomic magnetometers, for example optically pumped magnetometers, may conveniently be used. These are generally configured to operate at room temperature, so require no bulky cryogenic cooling, and the magnetic field sensing region of the sensor may be placed as close as a few millimetres from the scalp. Atomic magnetometers are commercially available as single compact packages with a footprint small enough to be able to easily space the sensors with centre points around 3 cm-5 cm apart.

In this way, the electrodes and sensors can be interspersed across the scalp. Typically a similar number of sensors and electrodes might be used, so if 32 electrodes are used, 32 sensors could also be used. Of course although shown as separate steps in FIG. 3, steps 110 and 120 may typically take place at the same time as a cap on which both sensors and electrodes are mounted is located on the subject's head. If carried out separately, steps 110 and 120 could take place in reverse order.

At least the sensors, and optionally both the sensors and electrodes may be mounted to a cap or other structure to assist accurate positioning and to avoid unwanted movement while in use. The cap could be moulded or printed using a fairly rigid plastic material, and could for example be shaped specifically for the head of the subject to be examined.

In step 130, probe electrical signals are applied to each of a plurality of combinations of the electrodes. These could be applied sequentially, or frequency division or other multiplexing techniques could be used to apply to some or all combinations at the same time. Different options for the probe electrical signals are provided elsewhere in this document.

In step 140, for each applied probe electrical signal, magnetic field signals arising from currents within the tissue due to the probe electrical signal are measured some or all of the magnetic field sensors. If frequency division multiplexing is used to apply probe signals to multiple electrode combinations at the same time, the resulting magnetic field signals arising from each probe electrical signal can be distinguished using corresponding frequency division demultiplexing.

At step 150, the collected magnetic field signals are used, with reference to or in combination with the applied probe electrical signals, to determine electrical properties within the subject tissue, for example within the brain. Typically, this may be carried out using tomographic inversion techniques to provide a map or image of a two dimensional slice or three dimensional volume of the tissue. Multiple such image frames may be determined sequentially. If frequency division multiplexing is used, frame rates of the order of 1 to 10 Hz are readily achievable.

Although particular embodiments of the invention have been described, it will be apparent to the skilled person that various modifications can be made without departing from the scope of the invention.

For example, although the described embodiments principally relate to detecting and/or monitoring electrical properties and/or changes in such properties with the human or animal brain or head, and/or to determining or measuring mechanisms, changes and/or pathologies within the head or brain, the techniques may be applied to other body regions such as the neck, chest or thorax, hips, limbs, and/or particular features, organs, structure or areas within these or elsewhere such as peripheral nerves, the spine, the lungs and so forth.

1. A non-invasive method of determining electrical properties within a brain of a human or animal subject, comprising:
disposing a plurality of electrodes across a scalp of the human or animal subject;
spacing a plurality of atomic magnetometer sensors around the scalp of the human or animal subject;
for each of a plurality of combinations of said electrodes, applying a probe electrical signal to the electrodes of said combination and measuring magnetic field signals arising from the probe electrical signals at each of a plurality of the atomic magnetometer sensors; and
using the magnetic field signals that were measured to determine electrical properties within the brain.

2. The method of claim 1, wherein the atomic magnetometer sensors are optically pumped magnetometers.

3. The method of claim 1, wherein the plurality of atomic magnetometer sensors comprises at least 16 such sensors.

4. The method of claim 1, wherein each atomic magnetometer sensor is spaced no more than 1 cm from a surface of the scalp.

5. The method of claim 1, wherein the atomic magnetometer sensors are mounted to a cap configured to fit on the scalp, and
wherein the electrodes are also mounted to the cap.

6. The method of claim 1, wherein each probe electrical signal has a frequency in a range from 100 Hz to 15 kHz, or in the range from 1.0 kHz to 2.5 kHz.

7. The method of claim 6, wherein the electrical properties that were determined comprise properties representing neuronal depolarisation within the brain.

8. The method of claim 6 further comprising:
providing a repeated stimulation to the human or animal subject, and for each stimulation applying a probe electrical signal to one of the combinations of electrodes and measuring the magnetic field signals arising from the probe electrical signal that was applied.

9. The method of claim 1, wherein each probe electrical signal has a frequency in a range from 100 Hz to 100 kHz.

10. The method of claim 9, wherein a time resolution of the electrical properties that were determined is between 0.1 and 10 seconds, or between 0.5 and 5 seconds.

11. The method of claim 10, wherein probe electrical signals are applied to two or more of said combinations of electrodes simultaneously, the simultaneous probe electrical signals having different frequencies, and distinguishing resulting magnetic field signals using the different frequencies.

12. The method of claim 9, wherein the determined electrical properties comprise properties representing one or more of: blood flow changes for example related to cerebral activity or pathology such as stroke, spontaneous normal brain function, or epileptic seizure.

13. The method of claim 1, wherein the electrical properties that were determined comprise properties representing brain pathology such as stroke or tumour.

14. The method of claim 1, further comprising:
carrying out a tomographic inversion of the magnetic field signals to determine a map or image of the electrical properties.

15. The method of claim 1, wherein the electrical properties are measures of impedance.

16. Apparatus for carrying out non-invasive determination of electrical properties within a brain of a human or animal subject, comprising:
a plurality of electrodes for applying probe electrical signals to a scalp of the human or animal subject;
a plurality of atomic magnetometer sensors arranged to fit around the scalp of the human or animal subject so as to measure magnetic field signals arising from the probe electrical signals; and
a probe signal source arranged, for each of a plurality of combinations of said electrodes, to apply a probe electrical signal to the electrodes of said combination.

17. The apparatus of claim 16 further comprising:
a tomographic inverter arranged to carry out a tomographic inversion of the measured magnetic field signals to determine the electrical properties.

18. The apparatus of claim 16 wherein the atomic magnetometer sensors are optically pumped magnetometer sensors.

19. The apparatus of claim 16, wherein the atomic magnetometer sensors are arranged so as to be spaced no more than 1 cm from a surface of the scalp.

20. The apparatus of claim 16, further comprising:
a cap configured to fit on the scalp,
wherein each atomic magnetometer sensor is mounted to the cap, and
wherein the electrodes are also mounted to the cap.

21. A method of carrying out electrical impedance tomography on a human or animal subject, comprising:
applying probe electrical signals to the human or animal subject using a plurality of contact electrodes such that imposed electrical currents flow within the human or animal subject, and
detecting magnetic fields arising from the imposed electrical currents using a plurality of atomic magnetometers disposed about the human or animal subject.

22. The method of claim 21 wherein the plurality of contact electrodes comprise at least twenty contact electrodes, and the plurality of atomic magnetometers comprise at least twenty atomic magnetometers interspersed with the contact electrodes.

23. The method of claim 1, further comprising:
imaging one or more of fast neuron activity, blood flow changes, epileptic seizure, or pathologies such as stroke or tumour, within the brain of a human or animal subject, by using the electrical properties that were determined.
